(19) United States
(12) Patent Application Publication
(10) Pub. No.: US 2012/0046539 A1
(43) Pub. Date: Feb. 23, 2012

(54) DUAL-CONTRAST MR IMAGING USING FLUID-ATTENUATION INVERSION RECOVERY (FLAIR)

(75) Inventor: Frederik Visser, Eindhoven (NL)

(73) Assignee: KONINKLIJKE PHILIPS ELECTRONICS N.V., EINDHOVEN (NL)

(21) Appl. No.: 13/258,605

(22) PCT Filed: Mar. 25, 2010
(86) PCT No.: PCT/IB2010/051303

§ 371 Date: Sep. 22, 2011

(30) Foreign Application Priority Data
Apr. 1, 2009 (EP) 09157062.2

(51) Int. Cl. A61B 5/055

(52) U.S. Cl. 600/410

(57) ABSTRACT

The invention relates to a method of MR imaging of at least a portion of a body (10) of a patient placed in an examination volume of an MR device (1). The acquisition of high-resolution three-dimensional FLAIR images as well as T2-weighted images at high main magnetic field strength (>3 Tesla) results in unacceptable long scan times. The present invention contemplates a new and improved MR imaging method which overcomes this problem. The method of the invention comprises the steps of subjecting the portion of the body (10) to a first imaging sequence (S1) for acquiring a first signal data set; immediately subsequent to the first imaging sequence (S1) subjecting the portion of the body (10) to an inversion RF pulse that inverses longitudinal magnetization within the portion; after an inversion delay period (TI) subjecting the portion of the body (10) to a second imaging sequence (S2) for acquiring a second signal data set; reconstructing first and second MR images from the first and second signal data sets respectively.

### FIELD OF THE INVENTION

The invention relates to the field of magnetic resonance (MR) imaging. It concerns a method of MR imaging of at least a portion of a body of a patient placed in an examination volume of an MR device. The invention also relates to an MR device and to a computer program to be run on an MR device.

### BACKGROUND OF THE INVENTION

Image-forming MR methods which utilize the interaction between magnetic fields and nuclear spins in order to form two-dimensional or three-dimensional images are widely used nowadays, notably in the field of medical diagnostics, because for the imaging of soft tissue they are superior to other imaging methods in many respects, do not require ionizing radiation and are usually not invasive.

According to the MR method in general, the body of the patient to be examined is arranged in a strong, uniform magnetic field whose direction at the same time defines an axis (normally the z-axis) of the co-ordinate system on which the measurement is based. The magnetic field produces different energy levels for the individual nuclear spins in dependence on the magnetic field strength which can be excited (spin resonance) by application of an electromagnetic alternating field (RF field) of defined frequency (so-called Larmor frequency, or MR frequency). From a macroscopic point of view the distribution of the individual nuclear spins produces an overall magnetization which can be deflected out of the state of equilibrium by application of an electromagnetic pulse of appropriate frequency (RF pulse) while the magnetic field extends perpendicular to the z-axis, so that the magnetization performs a precessional motion about the z-axis. The precessional motion describes a surface of a cone whose angle of aperture is referred to as flip angle. The magnitude of the flip angle is dependent on the strength and the duration of the applied electromagnetic pulse. In the case of a so-called 90° pulse, the spins are deflected from the z axis to the transverse plane (flip angle 90°).

After termination of the RF pulse, the magnetization relaxes back to the original state of equilibrium, in which the magnetization in the z direction is built up again with a first time constant T₁ (spin lattice or longitudinal relaxation time), and the magnetization in the direction perpendicular to the z direction relaxes with a second time constant T₂ (spin-spin or transverse relaxation time). The variation of the magnetization can be detected by means of receiving RF coils which are arranged and oriented within an examination volume of the MR device in such a manner that the variation of the magnetization is measured in the direction perpendicular to the z-axis. The decay of the transverse magnetization is accompanied, after application of, for example, a 90° pulse, by a transition of the nuclear spins (induced by local magnetic field inhomogeneities) from an ordered state with the same phase to a state in which all phase angles are uniformly distributed (dephasing). The dephasing can be compensated by means of a refocusing pulse (for example a 180° pulse). This produces an echo signal (spin echo) in the receiving coils.

In order to realize spatial resolution in the body, linear magnetic field gradients extending along the three main axes are superposed on the uniform magnetic field, leading to a linear spatial dependency of the spin resonance frequency. The signal picked up in the receiving coils then contains components of different frequencies which can be associated with different locations in the body. The signal data obtained via the receiving coils corresponds to the spatial frequency domain and is called k-space data. The k-space data usually includes multiple lines acquired with different phase encoding. Each line is digitized by collecting a number of samples. A set of k-space data is converted to an MR image by means of Fourier transformation.

Fluid-attenuation inversion recovery (FLAIR) is a popular MR imaging technique employed to suppress unwanted signal from fluid near or around tissue that an operator of an MR device wishes to visualize. It has been found particularly useful in brain and spinal imaging where brain tissue (grey and white matter) or spinal tissue is of interest and MR signals from surrounding cerebral spinal fluid (CSF) is undesirable. FLAIR pulse sequences are commonly used to provide improved conspicuity of lesions located in regions of the tissue near CSF.

Where FLAIR is used to evaluate abnormalities in the brain and spine, suppression of the CSF in the images is commonly desired so that contrast differences in lesions, tumors, and edema in tissue proximal to the CSF will be enhanced. The application and timing of an inversion recovery (IR) RF pulse determines the contrast that is produced during a FLAIR acquisition. FLAIR sequences that apply spatially selective IR RF pulses may exhibit problematic in-flow artifacts produced by CSF motion. As an alternative, non-selective FLAIR was developed. In non-selective FLAIR, a non-selective IR RF pulse that excites the entire region is applied before the actual imaging sequence is initiated. Different substances (tissue types) which have different relaxation characteristics will produce different levels of MR signal amplitude depending on the duration of an inversion delay period between the IR pulse and the instant at which the imaging sequence begins and the signal data sets for image reconstruction are acquired. In order to suppress MR signal contribution from CSF, usually the image acquisition should take place at the instant of the zero crossing of the longitudinal magnetization of CSF. In multi slice FLAIR, however, image contrast is often not as consistent through the image slices depending on the exact delay between the IR pulse and the acquisition of the respective image.

Implementations of three-dimensional FLAIR with non-selective inversion in which the problems of CSF-inflow artifacts and partial volume effects have been reduced are known in the art (see, e.g., U.S. Pat. No. 6,486,667). A drawback of these known techniques is that they work well at a main field strength of up to 3 Tesla. At higher fields, such as, e.g., 7 Tesla, the implementation of FLAIR is less straightforward due to specific absorption rate (SAR) constraints, high sensitivity to susceptibility, short T₂^{*} components and RF inhomogeneity. Moreover, the lengthening of T₁ relaxation times of grey and white matter, while T₁ of CSF is less field dependent, introduces more T₁-weighting of MR signals from grey and white matter, thereby compromising the desired T₂ contrast.

The FLAIR sequence together with the regular T₂-weighted turbo spin echo (TSE) sequence (i.e. without fluid-attenuation) are the most important techniques in neuro-radiology. However, a disadvantage of the known three-dimensional TSE techniques with isotropic voxel size <1 mm is the long scan time. High parallel imaging factors (SENSE, SMASH, see Pruessmann et al., “SENSE: Sensitivity Encoding for Fast MRI”, Magnetic Resonance in Medicine 1999, 42 (5), 1952-1962, and Sodickson et al., “Simultaneous acquisition of spatial harmonics (SMASH): Fast imaging with radio frequency coil arrays”, Magnetic Resonance in Medicine 1997, 38, 591-603) have been proposed to accelerate image acquisition, but the acquisition of a high-resolution three-dimensional T₂-weighted image and a corresponding FLAIR image from the same patient still results in unacceptable long scan times.

### SUMMARY OF THE INVENTION

The present invention contemplates a new and improved MR imaging method which overcomes the above-mentioned drawbacks and problems.

In accordance with the invention, a method of MR imaging of at least a portion of a body of a patient placed in an examination volume of an MR device is disclosed. The method of the invention comprises the following steps:
subjecting the portion of the body to a first imaging sequence for acquiring a first signal data set;
immediately subsequent to the first imaging sequence subjecting the portion of the body to an inversion RF pulse that inverses longitudinal magnetization within the portion;
after an inversion delay period subjecting the portion of the body to a second imaging sequence for acquiring a second signal data set;
reconstructing first and second MR images from the first and second signal data sets respectively.

The gist of the invention is the production of two images, such as, e.g., a three-dimensional T₂-weighted image (reconstructed from the first signal data set) and a FLAIR image (reconstructed from the second signal data set), within the scan time of a single conventional three-dimensional FLAIR experiment. The approach of the invention is actually a dual-contrast imaging sequence that begins with a regular image acquisition step (the first imaging sequence), i.e. without fluid-attenuation, immediately followed by an IR RF pulse that inverts the longitudinal magnetization existing after the first imaging sequence. The FLAIR image is then acquired in the second step after the appropriately selected inversion delay period. The overall scan time for the acquisition of both the (non fluid-attenuated) T₂-weighted image and the FLAIR image is not longer than the time required for acquisition of only the FLAIR image in the conventional manner.

According to a preferred embodiment of the invention, the examined portion of the body comprises at least two substances (such as, e.g., brain tissue and CSF) having different longitudinal relaxation times, the inversion delay period being selected such that the longitudinal magnetization of at least one of the substances (e.g. CSF) is essentially zero at the beginning of the second imaging sequence. This corresponds to the general FLAIR approach.

Moreover, the substances may also have different transverse relaxation times. In this case the duration of the first imaging sequence can be selected such the transverse magnetization of at least one of the substances (e.g. brain tissue) is essentially zero at the end of the first imaging sequence while the transverse magnetization of at least one other substance (e.g. CSF) is different from zero. The remaining transverse magnetization of CSF can be converted into longitudinal magnetization at the end of the first imaging sequence by means of a driven equilibrium (DRIVE) pulse, i.e. immediately before the IR RF pulse is irradiated. The short T₂ components (e.g. brain tissue) will not contribute to the longitudinal magnetization after the DRIVE pulse. In this embodiment, the first imaging sequence of the invention has the effect of a magnetization preparation sequence that saturates short T₂ components. In contrast to conventional FLAIR, the recovery of longitudinal magnetization of short T₂ substances (e.g. brain tissue) thus starts from zero after the irradiation of the IR RF pulse in accordance with the invention. This has a positive effect on T₂ contrast in the second imaging step at high main field strengths (e.g. 7 Tesla), where the longitudinal relaxation time of brain tissue is significantly increased (resulting in a corresponding reduction of the difference in longitudinal relaxation time between CSF and brain tissue). An increase of the signal-to-noise ratio (SNR) of 20-40% together with an increase of the contrast-to-noise ratio (CNR) between grey matter and white matter may be expected by the approach of the invention in comparison to conventional FLAIR imaging.

According to a further preferred embodiment of the invention, the inversion RF pulse is a spatially non-selective adiabatic inversion pulse. Undesirable CSF inflow effects can be avoided in this way, as explained above. Moreover, the adiabatic IR RF pulse is advantageous since it is insensitive to B₁ inhomogeneity, which is an issue at a high main magnetic field strength.

The method of the invention described thus far can be carried out by means of an MR device including at least one main magnet coil for generating a uniform steady magnetic field within an examination volume, a number of gradient coils for generating switched magnetic field gradients in different spatial directions within the examination volume, at least one RF coil, for generating RF pulses within the examination volume and/or for receiving MR signals from a body of a patient positioned in the examination volume, a control unit for controlling the temporal succession of RF pulses and switched magnetic field gradients, a reconstruction unit, and a visualization unit. The method of the invention is implemented by a corresponding programming of the reconstruction unit, the visualization unit, and/or the control unit of the MR device.

The method of the invention can be advantageously carried out in most MR devices in clinical use at present. To this end it is merely necessary to utilize a computer program by which the MR device is controlled such that it performs the above-explained method steps of the invention. The computer program may be present either on a data carrier or be present in a data network so as to be downloaded for installation in the control unit of the MR device.

### BRIEF DESCRIPTION OF THE DRAWINGS

The enclosed drawings disclose preferred embodiments of the present invention. It should be understood, however, that the drawings are designed for the purpose of illustration only and not as a definition of the limits of the invention. In the drawings:

FIG. 1 shows an MR device for carrying out the method of the invention;

FIG. 2 shows a diagram of the imaging sequence in accordance with the invention, together with a diagram showing the recovery of longitudinal magnetization during the inversion delay period.

### DETAILED DESCRIPTION OF EMBODIMENTS

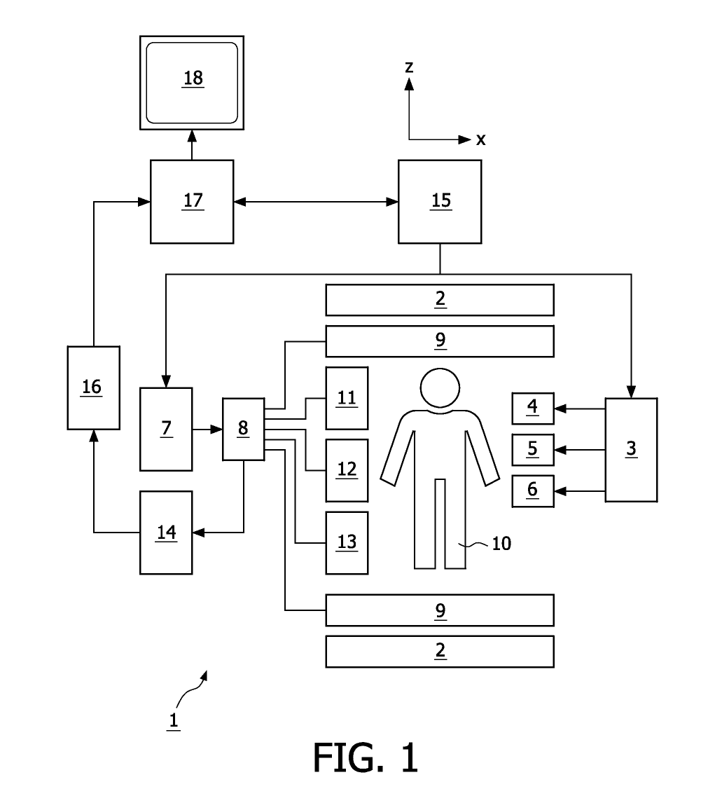

With reference to FIG. 1, an MR device 1 is shown. The device comprises superconducting or resistive main magnet coils 2 such that a substantially uniform, temporally constant main magnetic field is created along a z-axis through an examination volume.

A magnetic resonance generation and manipulation system applies a series of RF pulses and switched magnetic field gradients to invert or excite nuclear magnetic spins, induce magnetic resonance, refocus magnetic resonance, manipulate magnetic resonance, spatially and otherwise encode the magnetic resonance, saturate spins, and the like to perform MR imaging.

Most specifically, a gradient pulse amplifier 3 applies current pulses to selected ones of whole-body gradient coils 4, 5 and 6 along x, y and z-axes of the examination volume. A digital RF frequency transmitter 7 transmits RF pulses or pulse packets, via a send-/receive switch 8, to a whole-body volume RF coil 9 to transmit RF pulses into the examination volume. A typical MR imaging sequence is composed of a packet of RF pulse segments of short duration which taken together with each other and any applied magnetic field gradients achieve a selected manipulation of nuclear magnetic resonance. The RF pulses are used to saturate, excite resonance, invert magnetization, refocus resonance, or manipulate resonance and select a portion of a body 10 positioned in the examination volume. The MR signals are also picked up by the whole-body volume RF coil 9.

For generation of MR images of limited regions of the body 10 by means of parallel imaging, a set of local array RF coils 11, 12, 13 are placed contiguous to the region selected for imaging. The array coils 11, 12, 13 can be used to receive MR signals induced by body-coil RF transmissions.

The resultant MR signals are picked up by the whole body volume RF coil 9 and/or by the array RF coils 11, 12, 13 are demodulated by a receiver 14 preferably including a preamplifier (not shown). The receiver 14 is connected to the RF coils 9, 11, 12 and 13 via send-/receive switch 8.

A host computer 15 controls the gradient pulse amplifier 3 and the transmitter 7 to generate any of a plurality of MR imaging sequences, such as turbo spin echo (TSE) imaging, and the like. For the selected sequence, the receiver 14 receives a single or a plurality of MR data lines in rapid succession following each RF excitation pulse. A data acquisition system 16 performs analog-to-digital conversion of the received signals and converts each MR data line to a digital format suitable for further processing. In modern MR devices the data acquisition system 16 is a separate computer which is specialized in acquisition of raw image data.

Ultimately, the digital raw image data is reconstructed into an image representation by a reconstruction processor 17 which applies a Fourier transform or other appropriate reconstruction algorithms, such like SENSE or SMASH. The MR image may represent a planar slice through the patient, an array of parallel planar slices, a three-dimensional volume, or the like. The image is then stored in an image memory where it may be accessed for converting slices, projections, or other portions of the image representation into appropriate format for visualization, for example via a video monitor 18 which provides a man-readable display of the resultant MR image.

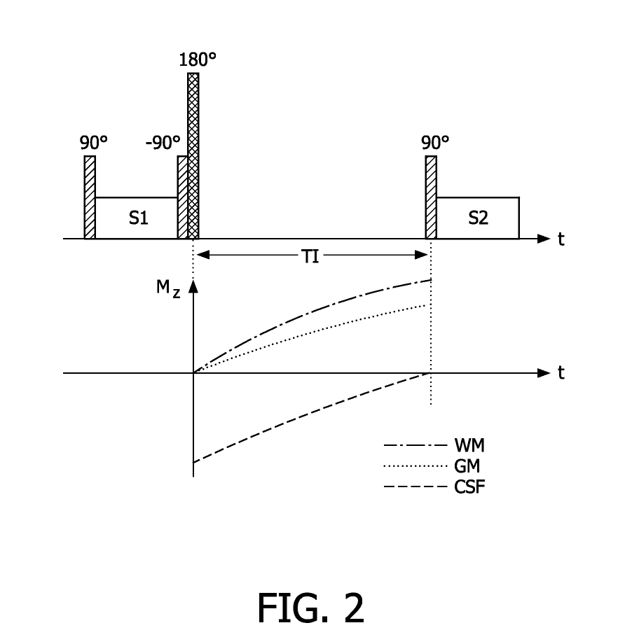

With continuing reference to FIG. 1 and with further reference to FIG. 2, an embodiment of the dual contrast imaging approach of the invention is explained which is used for brain imaging. The sequence shown in the upper diagram begins with a 90° RF pulse for excitation of magnetization, generated via the volume RF coil 9, followed by a first imaging sequence 51 during which a first signal data set is acquired. Sequence 51 is a three-dimensional TSE readout with advanced refocus pulse angle sweep (see Hennig et al., “Multi Echo Sequences with Variable Refocusing Flip Angles: Optimization of Signal Behavior Using Smooth Transitions Between Pseudo Steady States (TRAPS)”, Magnetic Resonance in Medicine 2003, 49, 527-535). A T₂-weighted image is reconstructed from the first signal data set. A −90° DRIVE pulse is irradiated at the end of the first imaging readout S1 in order to reset the remaining transverse magnetization of CSF back to the longitudinal axis. Very little transverse magnetization of short T₂ components (grey and white matter) remains at the end of sequence S1 to be transformed into longitudinal magnetization by the DRIVE pulse. The sequence comprising the initial 90° RF pulse, the TSE readout S1 and the DRIVE pulse thus behaves like a saturation preparation for these components. A non-selective adiabatic 180° inversion pulse is generated immediately after the DRIVE pulse. The 180° inversion pulse is optimized to meet the adiabatic conditions at the respective main magnetic field strength. The lower diagram in FIG. 2 shows the recovery of the longitudinal magnetization M_{z} of CSF and grey and white matter (GM, WM) as function of time t during the inversion delay period TI after the 180° inversion pulse. The magnetization of CSF starts at a large negative value while the longitudinal magnetization of GM and WM is essentially zero immediately after the 180° inversion pulse. A second 90° excitation pulse is irradiated after the inversion delay period TI, i.e. when the longitudinal magnetization of CSF is essentially zero and the longitudinal magnetization of GM and WM has recovered to a substantial positive value. The second 90° excitation pulse is followed by a second three-dimensional TSE readout S2 during which a second signal data set is acquired. A FLAIR image is reconstructed from this second signal data set.

The sequence shown in FIG. 2 can be used for dual contrast three-dimensional TSE imaging with T₂-weighting in the first readout S1 and FLAIR contrast in the second readout S2. The overall acquisition time is essentially not longer than the acquisition time of a conventional FLAIR imaging experiment. A further advantage of the shown sequence is that it produces an improved SNR and CNR in the second readout S2 due to saturation of short T₂ components after the first readout S1. It has to be noted, however, that the dual contrast approach of the invention can also be applied to two-dimensional and multi slice applications in case a full three-dimensional examination would result, after all, in a prohibitively long acquisition time.

1. Method of MR imaging of at least a portion of a body of a patient placed in an examination volume of an MR device, the method comprising the steps of
subjecting the portion of the body to a first imaging sequence for acquiring a first signal data set;
immediately subsequent to the first imaging sequence subjecting the portion of the body to an inversion RF pulse that inverses longitudinal magnetization within the portion;
after an inversion delay period subjecting the portion of the body to a second imaging sequence for acquiring a second signal data set;
reconstructing first and second MR images from the first and second signal data sets respectively.

2. Method of claim 1, wherein the first signal data set is T₂-weighted.

3. Method of claim 1, wherein the portion of the body comprises at least two substances having different longitudinal relaxation times, the inversion delay period being selected such that the longitudinal magnetization of at least one of the substances is essentially zero at the beginning of the second imaging sequence.

4. Method of claim 3, wherein the substances further have different transverse relaxation times, the duration of the first imaging sequence being selected such the transverse magnetization of at least one of the substances is essentially zero at the end of the first imaging sequence while the transverse magnetization of at least one other substance is different from zero.

5. Method of claim 1, wherein a driven equilibrium RF pulse is applied at the end of the first imaging sequence.

6. Method of claim 1, wherein the first and second imaging sequences are turbo spin echo sequences.

7. Method of claim 1, wherein the inversion RF pulse is a spatially non-selective adiabatic inversion pulse.

8. MR device for carrying out the method claimed in claim 1, which MR device includes at least one main magnet coil for generating a uniform, steady magnetic field within an examination volume, a number of gradient coils for generating switched magnetic field gradients in different spatial directions within the examination volume, at least one RF coil for generating RF pulses within the examination volume and/or for receiving MR signals from a body of a patient positioned in the examination volume, a control unit for controlling the temporal succession of RF pulses and switched magnetic field gradients, and a reconstruction unit, wherein the MR device is arranged to perform the following steps:
subjecting the portion of the body to a first imaging sequence for acquiring a first signal data set, the first imaging sequence comprising RF pulses, generated via the RF coil, and switched magnetic field gradients, generated via the gradient coils;
immediately subsequent to the first imaging sequence subjecting the portion of the body to an inversion RF pulse, generated via the RF coil, that inverses longitudinal magnetization within the portion;
after an inversion delay period subjecting the portion of the body to a second imaging sequence for acquiring a second signal data set, the second imaging sequence comprising RF pulses, generated via the RF coil, and switched magnetic field gradients, generated via the gradient coils;
reconstructing, by means of the reconstruction unit, first and second MR images from the first and second signal data sets respectively.

9. MR device of claim 8, further comprising a set of array RF coils for receiving MR signals from the body in parallel.

10. Computer program to be run on an MR device, which computer program comprises instructions for:
generating a first imaging sequence for acquiring a first signal data set;
immediately subsequent to the first imaging sequence generating an inversion RF pulse;
after an inversion delay period generating a second imaging sequence for acquiring a second signal data set;
reconstructing first and second MR images from the first and second signal data sets respectively.
